(19) United States
(12) Patent Application Publication
(10) Pub. No.: US 2025/0255890 A1
(43) Pub. Date: Aug. 14, 2025

(54) PSILOCYBIN CULTIVATION, EXTRACTION, AND THERAPEUTIC USE

(71) Applicant: Golden Fleece Research Group Inc., Cerritos, CA (US)

(72) Inventors: Jason Carbajal, Cerritos, CA (US); Serob Minasyan, Cerritos, CA (US)

(21) Appl. No.: 19/053,157

(22) Filed: Feb. 13, 2025

Related U.S. Application Data
(60) Provisional application No. 63/553,085, filed on Feb. 13, 2024.

(51) Int. Cl. A61K 31/675; A01G 18/20; A01G 18/50; A61K 31/455; A61K 36/07

(52) U.S. Cl. CPC A61K 31/675; A01G 18/20; A01G 18/50; A61K 31/455; A61K 36/078; A61K 2236/15; A61K 2236/33; A61K 2236/50; A61K 2236/51

(57) ABSTRACT

Provided herein are methods for treating a patient with ADHD and anxiety, comprising administering to the patient a therapeutically effective amount of psilocybin. The disclosure also provides methods for treating ADD and reducing symptoms of Tourette Syndrome using psilocybin. Additionally, methods of cultivation, extraction, and purification of psilocybin mushrooms are described. The psilocybin is obtained from mushroom species such as Psilocybe cubensis, Psilocybe cyanescens, Psilocybe semilanceata, or Psilocybe azurescens. The methods involve tissue culture, agar inoculation, grain-to-grain transfer, bulk substrate inoculation, and fruiting. Extraction and purification steps include harvesting, drying, grinding, solvent extraction, filtration, evaporation, and cryogenic separation.

### CROSS-REFERENCE TO RELATED APPLICATIONS

This application claims priority from U.S. Provisional Application Ser. No. 63/553,085 filed on Feb. 13, 2024, which is incorporated herein by reference in its entirety.

### STATEMENT REGARDING FEDERALLY SPONSORED RESEARCH OR DEVELOPMENT

Not Applicable.

### FIELD

The present disclosure relates to psilocybin mushrooms, and more particularly to methods of cultivation, extraction, and purification of psilocybin mushrooms and the use of psilocybin in the treatment of mental disorders and neurological diseases.

### INTRODUCTION

Psilocybin mushrooms, also known as “magic mushrooms,” contain naturally occurring psychedelic compounds, primarily psilocybin. In recent years, there has been growing interest in exploring the potential therapeutic applications of psilocybin for various psychiatric and behavioral disorders. Hundreds of psilocybin-containing mushroom species have been identified worldwide, with some of the most common and widespread species including Psilocybe cubensis, Psilocybe cyanescens, Psilocybe semilanceata, and Psilocybe azurescens.

When consumed, psilocybin can induce hallucinogenic or psychedelic effects. However, the specific psychoactive effects and potential medicinal uses may vary depending on factors such as the type of psilocybin mushroom, extraction and purification processes, and the strength of different strains. This variability has led to increased research efforts aimed at understanding how these factors influence the effects and potential therapeutic applications of psilocybin.

The cultivation of psilocybin mushrooms typically involves several steps, including isolation of a clean culture, inoculation and growth on agar medium, subculturing, grain-to-grain transfer, bulk substrate inoculation, and providing proper fruiting conditions. The extraction and purification process generally includes harvesting, drying, grinding, solvent extraction, filtration, evaporation, and further purification steps.

In addition to psilocybin, magic mushrooms contain other alkaloids in minor amounts, such as psilocin, baeocystin, and norbaeocystin. These compounds may contribute to the overall psychoactive effects or potentially induce synergistic effects with psilocybin. The presence and content of these alkaloids in psilocybin products derived from different processes or mushroom species may be relevant to their psychoactivity and potential medicinal applications.

Research into the therapeutic potential of psilocybin has expanded to include various neurological and mental disorders. Some areas of investigation include the treatment of anxiety, depression, addiction, and other psychiatric conditions. However, further research is needed to fully understand the efficacy, safety, and optimal use of psilocybin in clinical settings.

As interest in the potential therapeutic applications of psilocybin continues to grow, there is a need for improved methods of cultivation, extraction, and purification, as well as a better understanding of how different psilocybin products may be used to address specific mental health conditions.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a method for treating a patient with ADHD and anxiety is provided. The method includes administering to the patient a therapeutically effective amount of psilocybin.

According to another aspect of the present disclosure, a method for treating a patient with ADD is provided. The method includes administering to the patient a therapeutically effective amount of psilocybin.

According to a further aspect of the present disclosure, a method for reducing the symptoms of a patient with Tourette Syndrome is provided. The method includes administering to the patient a therapeutically effective amount of psilocybin.

The foregoing general description of the illustrative embodiments and the following detailed description thereof are merely exemplary aspects of the teachings of this disclosure and are not restrictive.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure relates to methods for cultivating, extracting, and purifying psilocybin-containing mushrooms, as well as therapeutic applications of the resulting psilocybin products. Psilocybin mushrooms, also known as “magic mushrooms,” contain naturally occurring psychedelic compounds, primarily psilocybin. Various species of psilocybin-containing mushrooms may be utilized in the methods described herein, including but not limited to Psilocybe cubensis, Psilocybe cyanescens, Psilocybe semilanceata, and Psilocybe azurescens.

The methods disclosed encompass cultivation techniques for growing psilocybin mushrooms under controlled conditions, as well as extraction and purification processes to isolate and concentrate the active compounds. These processes may be tailored to optimize the yield and purity of psilocybin and related compounds from different mushroom species.

Additionally, the disclosure provides methods for using the purified psilocybin products in therapeutic applications. These applications may include the treatment of various neurological and mental health conditions. The methods described herein may allow for the development of standardized psilocybin products with consistent potency and composition, which may be advantageous for therapeutic use.

The following detailed description provides further information on the cultivation, extraction, purification, and therapeutic use of psilocybin and psilocybin-containing mushrooms.

### Cultivation of Psilocybin Mushrooms

The cultivation of psilocybin mushrooms may involve several steps to ensure proper growth and development. In some cases, the process begins with isolation, where a clean culture is obtained from a spore print or spore syringe. The isolated culture may then be transferred to an agar medium for agar inoculation, allowing for the growth and isolation of mycelium.

In some implementations, the agar medium may contain agar, malt extract, peptone, and water. The specific composition of the agar medium may vary depending on the particular strain of psilocybin mushroom being cultivated.

Following agar inoculation, a subculture step may be performed. This step may involve transferring healthy mycelium to fresh agar for further propagation. The subculture process may be repeated multiple times to ensure a robust and contamination-free mycelial culture.

Once a strong mycelial culture is established, a grain-to-grain transfer may be performed. This step may involve inoculating sterilized grain with the mycelium. In some cases, the grain spawn medium may contain grains such as rye or millet, water, and gypsum. The specific composition of the grain spawn medium may be adjusted based on the nutritional requirements of the particular psilocybin mushroom strain being cultivated.

Following successful colonization of the grain spawn, a bulk substrate inoculation may be performed. This step may involve mixing the colonized grain with a bulk substrate. In some implementations, the bulk substrate may contain vermiculite, brown rice flour, and water, providing a nutrient-rich environment for mushroom growth.

The final stage of cultivation may involve providing proper fruiting conditions. These conditions may include specific temperature ranges, humidity levels, and light exposure to promote the formation and maturation of mushroom fruiting bodies. The exact environmental parameters may vary depending on the specific psilocybin mushroom species being cultivated.

In some cases, the cultivation process may be optimized for different psilocybin mushroom species, such as Psilocybe cubensis, Psilocybe cyanescens, Psilocybe semilanceata, or Psilocybe azurescens. Each species may require slight modifications to the cultivation process or growth media composition to achieve optimal results.

The cultivation process may be carried out under controlled conditions to ensure consistency and minimize the risk of contamination. In some implementations, sterile techniques and equipment may be used throughout the cultivation process to maintain the purity of the psilocybin mushroom cultures.

### Extraction and Purification of Psilocybin

The extraction and purification of psilocybin from harvested mushrooms may involve several steps. In some cases, the process begins with drying the harvested mushrooms. The dried mushrooms may then be ground into a powder to increase surface area for extraction.

In some implementations, a solvent extraction method may be used. For example, methanol may be mixed with the powdered mushrooms in a 1:5 ratio. The mixture may then undergo ultrasound-assisted extraction, which may involve exposing the solution to ultrasonic waves at a frequency of 20 kHz for a duration of 30 minutes at a temperature of 60 degrees Celsius.

Following extraction, the solution may be filtered to remove solid particles. In some cases, vacuum filtration with a 3 micron filter may be employed for this step. The filtered solution may then be concentrated through evaporation, for example by using a rotary evaporator (rotovap) for 30 minutes.

Further purification steps may involve cryogenic freezing. In some implementations, the concentrated solution may be placed in an aluminum container and frozen at −20 degrees Celsius for 12 hours. This freezing process may be repeated multiple times, with accumulated material being removed after each freezing cycle.

The collected material from the freezing cycles may be combined and dried. In some cases, this drying step may involve placing the material in a vacuum oven at 110 degrees Fahrenheit for 10 hours.

In some implementations, the dried material may be further processed by mixing with niacin. For example, the dried material may be combined with niacin in a 100:5 ratio and placed in a sonicator for 25 minutes.

It should be understood that variations in the extraction and purification process may be made. For example, different solvents such as ethanol or water may be used for extraction. The specific parameters for ultrasound-assisted extraction, filtration, evaporation, and cryogenic freezing may be adjusted based on the particular mushroom species or desired end product.

The described extraction and purification process may result in a concentrated form of psilocybin and potentially other psychoactive compounds naturally present in the mushrooms. The final product may be suitable for further analysis, formulation, or potential therapeutic applications as described elsewhere in this disclosure.

### Therapeutic Applications of Psilocybin

Psilocybin may be used for treating various neurological and mental disorders. In some cases, psilocybin may be administered to patients with attention deficit hyperactivity disorder (ADHD), anxiety, attention deficit disorder (ADD), or Tourette Syndrome.

For ADHD and anxiety treatment, psilocybin may be administered in doses ranging from 10-25 mg. The psilocybin may be given orally in the form of capsules or tablets. In some cases, a lower initial dose of 5-10 mg may be used, with subsequent doses increased as needed based on patient response.

For ADD treatment, psilocybin dosing may range from 15-30 mg. The psilocybin may be administered sublingually as a liquid tincture or as an oral lozenge. Multiple smaller doses spread throughout the day may be used in some cases.

To reduce symptoms of Tourette Syndrome, psilocybin doses of 20-40 mg may be utilized. The psilocybin may be given as an oral solution or as a rapidly-dissolving tablet placed under the tongue. In some cases, dosing may occur 2-3 times per week.

Psilocybin may also have therapeutic applications for other conditions. In some cases, psilocybin may be used to treat depression, with doses ranging from 10-30 mg given orally once every 1-2 weeks. For anorexia treatment, psilocybin doses of 15-25 mg may be administered orally or sublingually on a weekly or biweekly basis.

In cases of alcohol use disorder, psilocybin doses of 25-40 mg may be given orally or sublingually, typically as part of a comprehensive treatment program. For smoking cessation, psilocybin doses of 20-30 mg may be administered 2-3 times over the course of several weeks, in conjunction with cognitive behavioral therapy.

The specific dosing and administration methods may vary based on individual patient factors. In all cases, psilocybin should be administered under medical supervision in a controlled setting. Careful monitoring of patient response and side effects may be necessary to determine optimal dosing regimens.

### Synergistic Effects of Psilocybin and Other Compounds

In some cases, psilocybin mushrooms may contain additional alkaloids beyond psilocybin itself. These additional compounds may include psilocin, baeocystin, norbaeocystin, and other unidentified bioactive substances. The presence and relative concentrations of these compounds may vary depending on the specific mushroom species and growing conditions.

Psilocin may be a metabolite of psilocybin that is formed in the body after ingestion. In some cases, psilocin may be directly responsible for the psychoactive effects associated with psilocybin mushrooms.

Baeocystin and norbaeocystin are structurally related to psilocybin but with slight molecular differences. The pharmacological effects of these compounds in humans are not fully characterized. In some cases, baeocystin and norbaeocystin may contribute to the overall effects experienced when consuming psilocybin mushrooms.

The combination of psilocybin with these additional compounds may potentially result in synergistic effects. Synergy refers to the phenomenon where the combined effect of multiple substances is greater than the sum of their individual effects. In some cases, the presence of compounds like baeocystin and norbaeocystin alongside psilocybin may modulate or enhance the therapeutic potential.

The specific interactions between psilocybin and these additional compounds are an area of ongoing research. In some cases, the entourage effect, a concept where multiple compounds work together to produce effects different from those of any single compound, may be relevant to the therapeutic applications of psilocybin mushrooms.

The relative ratios of psilocybin to other compounds may vary between different mushroom species and cultivation methods. In some cases, these variations may lead to differences in the overall effects or therapeutic potential of different psilocybin-containing preparations.

Understanding the roles of these additional compounds may be important for optimizing the therapeutic applications of psilocybin. In some cases, isolating pure psilocybin may not capture the full therapeutic potential that may be present in whole mushroom extracts or preparations that retain a broader spectrum of naturally occurring compounds.

Further research may be needed to fully elucidate the individual and combined effects of these various compounds found in psilocybin mushrooms. This understanding may inform the development of more targeted and effective therapeutic applications in the future.

### Integration of Cultivation, Extraction and Therapeutic Use

The integration of cultivation, extraction, and therapeutic use of psilocybin involves a coordinated process that aims to produce high-quality, consistent, and tailored products for specific medical applications.

In some cases, the cultivation process may be optimized to enhance the production of desired compounds. For example, certain growth conditions or substrate compositions may be selected to promote the synthesis of psilocybin or other beneficial alkaloids in the mushrooms. The selection of specific Psilocybe species or strains may also influence the alkaloid profile of the final product.

The extraction and purification processes may be adjusted based on the intended therapeutic use. In some implementations, a full-spectrum extract containing psilocybin along with other naturally occurring compounds may be desired. Alternatively, the purification process may be designed to isolate specific compounds or achieve a particular ratio of active ingredients.

The integration of these processes allows for the production of standardized psilocybin products with consistent potency and composition. This standardization may be crucial for conducting reliable clinical studies and ensuring reproducible therapeutic effects.

In some cases, the cultivation and extraction methods may be tailored to produce psilocybin products with specific alkaloid profiles. For example, a product intended for anxiety treatment may have a different composition than one designed for depression therapy. The ratio of psilocybin to other compounds such as baeocystin or norbaeocystin may be adjusted to potentially enhance therapeutic efficacy for particular indications.

The integration of cultivation and extraction with therapeutic use also involves considerations of dosage form and administration route. In some implementations, the final product may be formulated as a capsule, tablet, or liquid solution, depending on the intended method of administration and therapeutic application.

Quality control measures may be implemented throughout the integrated process to ensure the safety and efficacy of the final product. This may include testing for contaminants, verifying alkaloid content, and ensuring consistency between batches.

In some cases, the integrated approach allows for the development of personalized medicine. By carefully controlling the cultivation and extraction processes, it may be possible to produce psilocybin products tailored to individual patient needs or specific mental health conditions.

The integration of these processes may also facilitate the exploration of synergistic effects between psilocybin and other compounds present in the mushrooms. This holistic approach may lead to the development of more effective therapeutic formulations that leverage the full potential of the mushroom's natural composition.

### Method of Use

Provided herein are medicinal use of Psilocybin products from the described processes. The invention is to use the purified Psilocybin products to treat or reduce symptoms of various neurological and mental disorders.

In one aspect, psilocybin is effective in treating and reducing ADHD, anxiety, ADD and the symptoms of Tourette Syndrome. As shown in Table 1, an identified group of selected patients with certain neurological and mental disorders has demonstrated positive responses after intaking the psilocybin products. Some patients suffered significant side effects when taking prescription drugs for the respective indications. Psilocybin has shown significant therapeutic effects in reducing and treating anxiety, ADHD, ADD and Tourette Syndrome with minimal side effects.

TABLE 1
Therapeutic Effects of Psilocybin

| Individual | Age | Indications | Experiences* |
|---|---|---|---|
| Female | 28 | ADHD, Anxiety | Wave of calmness, |
| | | | boosted creativity |
| Male | 41 | ADD | More focused. Elevated creativity |
| Female | 15 | Tourette | Reduced major episodes, |
| | | Syndrome | improved sleeps |

*With psilocybin treatment

In another aspect, psilocybin is effective in reducing anxiety, depression, anorexia, alcohol use disorder, and assisting with smoking cessation.

A number of implementations have been described. Nevertheless, it will be understood that various modifications may be made without departing from the spirit and scope of the disclosure. Accordingly, other implementations are within the scope of the following claims.

### EXAMPLES

### Process of Cultivating, Extraction and Purification

The processes are exemplified by the following steps:

Tissue culture is taken from the fruit body and cultivated in tissue culture solution.

Fruit body is transferred to a grow median then harvested and dried and ground down and mixed with methanol 1:5 ratio

Solution is placed in ultrasound-assisted extraction at 20 kHz for 30 minutes at 60 degrees celsius.

Solution filtered through a vacuum filtration system with a 3 micron filter

The solution is then rotovapped for 30 minutes.

Solution is then placed in an aluminum container cryogenically frozen at −20 for 12 hours.

The solution is then removed where the first stage of separation occurs.

Remove accumulated material from aluminum container

Remaining solution is then placed back in the aluminum container cryogenically frozen again at −20 for 12 hours again

The solution is then removed and separates again

Remove accumulated material from the aluminum container

Remaining solution is then placed back in the aluminum container cryogenically frozen again at −20 for 12 hours again

The solution is then removed and separates again

Remove accumulated material from the aluminum container

The 3 materials are then combined and placed in a vacuum oven for 10 hours at 110 degrees fahrenheit.

Dried finished solution is then placed in a sonicator for 25 minutes with 100:5 niacin to dried material.

### Other Embodiments

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

### References Cited

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

What is claimed is:

1. A method for treating a neurological or mental disorder in a subject, comprising:
administering to the subject a therapeutically effective amount of a psilocybin composition derived from a Psilocybe mushroom species, wherein the psilocybin composition is obtained by a process comprising:
cultivating the Psilocybe mushroom species using a tissue culture technique;
extracting psilocybin from the cultivated mushrooms using a solvent extraction method; and
purifying the extracted psilocybin using cryogenic separation.

2. The method of claim 1, wherein the Psilocybe mushroom species is selected from the group consisting of Psilocybe cubensis, Psilocybe cyanescens, Psilocybe semilanceata, and Psilocybe azurescens.

3. The method of claim 1, wherein the tissue culture technique comprises:
isolating a clean culture from a spore print or spore syringe;
inoculating the culture onto an agar medium;
subculturing healthy mycelium onto fresh agar; and
transferring the mycelium to a grain spawn medium.

4. The method of claim 1, wherein the solvent extraction method comprises:
grinding dried mushrooms into a powder;
mixing the powder with methanol in a 1:5 ratio; and
subjecting the mixture to ultrasound-assisted extraction at 20 kHz for 30 minutes at 60 degrees Celsius.

5. The method of claim 1, wherein the cryogenic separation comprises:
freezing the extracted psilocybin solution at −20 degrees Celsius for 12 hours;
removing accumulated material; and
repeating the freezing and removal steps at least two additional times.

6. The method of claim 1, further comprising combining the purified psilocybin with niacin in a 100:5 ratio.

7. The method of claim 1, wherein the neurological or mental disorder is selected from the group consisting of attention deficit hyperactivity disorder (ADHD), anxiety, attention deficit disorder (ADD), and Tourette syndrome.

8. A method for cultivating and extracting psilocybin from Psilocybe mushrooms, comprising:
isolating a clean culture from a Psilocybe mushroom species;
propagating mycelium on an agar medium;
transferring the mycelium to a grain spawn medium;
inoculating a bulk substrate with the colonized grain;
providing fruiting conditions for mushroom growth;
harvesting and drying the mushrooms;
extracting psilocybin from the dried mushrooms using a solvent; and
purifying the extracted psilocybin using cryogenic separation.

9. The method of claim 8, wherein the Psilocybe mushroom species is selected from the group consisting of Psilocybe cubensis, Psilocybe cyanescens, Psilocybe semilanceata, and Psilocybe azurescens.

10. The method of claim 8, wherein the agar medium comprises malt extract, peptone, and water.

11. The method of claim 8, wherein the grain spawn medium comprises rye or millet grains.

12. The method of claim 8, wherein the bulk substrate comprises vermiculite, brown rice flour, and water.

13. The method of claim 8, wherein extracting psilocybin from the dried mushrooms comprises:
grinding the dried mushrooms into a powder;
mixing the powder with methanol in a 1:5 ratio; and
subjecting the mixture to ultrasound-assisted extraction at 20 kHz for 30 minutes at 60 degrees Celsius.

14. The method of claim 13, wherein purifying the extracted psilocybin using cryogenic separation comprises:
freezing the extracted psilocybin solution at −20 degrees Celsius for 12 hours;
removing accumulated material; and
repeating the freezing and removal steps at least two additional times.

15. A pharmaceutical composition for treating a neurological or mental disorder, comprising:
a therapeutically effective amount of psilocybin derived from a Psilocybe mushroom species; and
at least one pharmaceutically acceptable excipient,
wherein the psilocybin is obtained by a process comprising cultivation of the Psilocybe mushroom species, solvent extraction of psilocybin from the cultivated mushrooms, and cryogenic purification of the extracted psilocybin.

16. The pharmaceutical composition of claim 15, wherein the Psilocybe mushroom species is selected from the group consisting of Psilocybe cubensis, Psilocybe cyanescens, Psilocybe semilanceata, and Psilocybe azurescens.

17. The pharmaceutical composition of claim 15, wherein the solvent extraction comprises:
grinding dried mushrooms into a powder;
mixing the powder with methanol in a 1:5 ratio; and
subjecting the mixture to ultrasound-assisted extraction at 20 kHz for 30 minutes at 60 degrees Celsius.

18. The pharmaceutical composition of claim 15, wherein the cryogenic purification comprises:
freezing the extracted psilocybin solution at −20 degrees Celsius for 12 hours;
removing accumulated material; and
repeating the freezing and removal steps at least two additional times.

19. The pharmaceutical composition of claim 15, further comprising niacin in a 100:5 ratio with the psilocybin.

20. The pharmaceutical composition of claim 19, wherein the neurological or mental disorder is selected from the group consisting of attention deficit hyperactivity disorder (ADHD), anxiety, attention deficit disorder (ADD), and Tourette syndrome.
